(19) United States
(12) Patent Application Publication
(10) Pub. No.: US 2025/0115573 A1
(43) Pub. Date: Apr. 10, 2025

(54) PYRIDAZINE-BASED PYRIDINE DERIVATIVES AS BIOLOGICALLY ACTIVE AGENTS

(71) Applicants: Mohd Imran, Rafha (SA); Abida Ash Mohd, Rafha (SA); Hamdy Khamees Thabet, Rafha (SA); Md Afroz Bakht, Alkharj (SA); Syed Mohammed Basheeruddin Asdaq, Riyadh (SA); Naira Nayeem, Rafha (SA); Md Tauquir Alam, Rafha (SA); Muhammad Wahab Abjad, Pittsburgh, PA (US); Maria Abdul Ghafoor Raja, Pittsburgh, PA (US); AlAnood Waslullah T. AlHjajji, Makkah Al Mukarramah (SA)

(72) Inventors: Mohd Imran, Rafha (SA); Abida Ash Mohd, Rafha (SA); Hamdy Khamees Thabet, Rafha (SA); Md Afroz Bakht, Alkharj (SA); Syed Mohammed Basheeruddin Asdaq, Riyadh (SA); Naira Nayeem, Rafha (SA); Md Tauquir Alam, Rafha (SA); Muhammad Wahab Abjad, Pittsburgh, PA (US); Maria Abdul Ghafoor Raja, Pittsburgh, PA (US); AlAnood Waslullah T. AlHjajji, Makkah Al Mukarramah (SA)

(21) Appl. No.: 18/481,991

(22) Filed: Oct. 5, 2023

(51) Int. Cl. C07D 401/10; A61P 31/04; A61P 31/10; A61P 39/06

(52) U.S. Cl. CPC C07D 401/10; A61P 31/04; A61P 31/10; A61P 39/06

(57) ABSTRACT

The present disclosure relates to a compound of Formula I

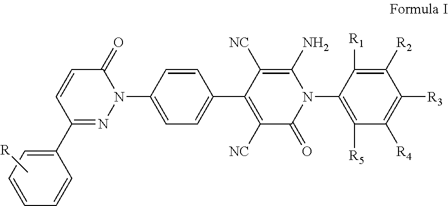

wherein R is selected from the group consisting of —H, —F, —Cl, —Br, —I, —NO₂, —CH₃, and —C₂H₅; R₁, R₂, R₃, R₄ and R₅ are independently selected from the group consisting of —H, —COOH, -, —CONH₂, —COOCH₃, —COOCH₂CH₃, —CN, —NO₂, —OH, —CF₃, —SO₂NH₂, —F, —Cl, —Br, —I, —OCH₃, —CH₃, and —COCH₃, or a pharmaceutically acceptable salt thereof.

### TECHNICAL FIELD

The present application relates to a chemical formulation. More particularly, the present application discloses pyridazine-based pyridine derivatives and/or a pharmaceutically acceptable salt thereof, as an antioxidant, COX-2 inhibitor and antimicrobial agent.

### BACKGROUND

Inflammation is an ordinary reaction of human tissues against harmful stimuli. The cyclooxygenase (COX) enzyme converts arachidonic acid into prostaglandin to produce inflammatory events. There are two types of COX, namely, COX-1 and COX-2. COX-2 is mainly responsible for inflammatory processes, wherein COX-1 maintains the normal work of a cell. Most existing non-steroidal anti-inflammatory drugs (NSAIDs), like indomethacin, are non-specific COX inhibitors because they inhibit COX-1 and COX-2. The inhibition of COX-1 leads to the development of gastric ulcerating effects, which is the main side effect of the existing NSAIDs. The specific COX-2 inhibitors also have associated side effects, for example, rofecoxib can cause cerebrovascular risk and cardiac toxicity in some patients at the standard dose.

Reactive oxidative stress (ROS) is implicated in the inflammatory process and the induction of gastric ulcers by NSAIDs. It is also assumed that oxidative stress in a cell is complementary to the inflammation process and vice versa. The ROS also delays the wound-healing process of the infected wounds. Antioxidant therapy is also dictated in the infected wound healing process.

Pyridazine and pyridine templates possess COX-2 inhibitory, antioxidant, and antimicrobial activity. However, pyridazine-based pyridine derivatives have not yet been reported as antioxidant, COX-2 inhibitors and antimicrobials.

One of the conventional US patent applications provides substituted 6-phenyl-3(2H)-pyridazinones. It also provides the preparation of 6-phenyl-4,5-dihydropyridazin-3(2H)-one and 6-phenylpyridazin-3(2H)-one. Another conventional prior art discloses a process of preparing 4-(6-oxo-3-phenylpyridazin -1(6H)-yl)benzaldehyde using 6-phenylpyridazin-3(2H)-one. For example, Molecules, 2020, 25, 2002, https://doi.org/10.3390/molecules25092002 and Drug Development Research, 2020, 1-12, https://doi.org/10.1002/ddr.21655. Such a prior art reports some hydrazones, thiazoles, and thiazolidinones as anti-inflammatory agents.

Other conventional US patent applications disclose the structures and the methods of preparation of various cyanoacetamide derivatives.

Therefore, the present disclosure provides pyridazine-based pyridine derivatives of Formula I, or a pharmaceutically acceptable salt thereof, as an antioxidant, COX-2 inhibitor and antimicrobial agent.

### SUMMARY

In an aspect of the present disclosure, pyridazine-based pyridine derivatives of Formula I, or a pharmaceutically acceptable salt thereof, as an antioxidant, COX-2 inhibitor and antimicrobial agent is disclosed.

In another aspect of the present disclosure, Formula I is disclosed as:

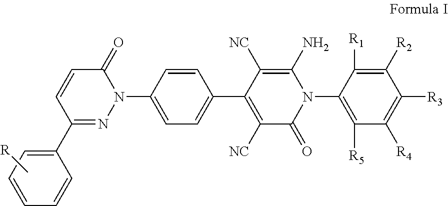

wherein R is selected from the group consisting of —H, —F, —Cl, —Br, —I, —NO₂, —CH₃, and —C₂H₅; R₁, R₂, R₃, R₄ and R₅ are independently selected from the group consisting of —H, —COOH, -, —CONH₂, —COOCH₃, —COOCH₂CH₃, —CN, —NO₂, —OH, —CF₃, —SO₂NH₂, —F, —Cl, —Br, —I, —OCH₃, —CH₃, and —COCH₃, or a pharmaceutically acceptable salt thereof.

In another aspect of the present disclosure, the compound is an active ingredient of a pharmaceutical composition for use in treating diseases associated with a higher level of ROS and COX-2. The compound is an antimicrobial agent.

In another aspect of the present disclosure, the compound of Formula I is administered to a subject through a route selected from one or more of oral, nasal, rectal, parenteral, intracisternal, intravaginal, intraperitoneal, topical and transdermal.

In another aspect of the present disclosure, the compound of Formula I is in a form selected from one or more of tablets, pills, capsules, powders, granules, ointments, parenteral solutions, suspensions, aerosols, sprays, drops, ampules, auto-injector devices, suppositories, oral solutions, intranasal dosage form, sublingual dosage form or rectal dosage form or dosage form for administration by inhalation or insufflation.

In another aspect of the present disclosure, the compound of Formula I is an inhibitor of ROS and COX-2. The diseases are selected from one or more groups consisting of ulcerative colitis, psoriasis, gout, osteoarthritis, ankylosing spondylitis, rheumatoid arthritis, pancreatitis, diabetes, hepatitis, irritable bowel diseases, asthma, kidney injury, ischemic stroke, atherosclerosis, neuropathic pain, epilepsy, depression, Parkinson's diseases, Crohn's disease, Alzheimer's diseases, and cancer.

In another embodiment of the present disclosure, a compound selected from the group consisting of 1-(4-Acetylphenyl)-6-amino-2-oxo-4-(4-(6-oxo-3-phenylpyridazin-1(6H)-yl)phenyl)-1,2-dihydropyridine-3,5-dicarbonitrile, 6-Amino -2-oxo-4-(4-(6-oxo-3-phenylpyridazin-1(6H)-yl)phenyl)-1-(p-tolyl)-1,2-dihydro -pyridine-3,5-dicarbonitrile, 6-Amino-1-(4-chlorophenyl)-2-oxo-4-(4-(6-oxo-3-phenylpyridazin-1(6H)-yl)phenyl)-1,2-dihydropyridine-3,5-dicarbonitrile, 6-Amino -1-(2-methoxyphenyl)-2-oxo-4-(4-(6-oxo-3-phenylpyridazin-1(6H)-yl)phenyl)-1,2-dihydropyridine-3,5-dicarbonitrile, Ethyl 4-(6-amino-3,5-dicyano-2-oxo-4-(4-(6-oxo-3-phenylpyridazin-1(6H)-yl)phenyl)pyridin-1(2H)-yl)benzoate, 6-Amino-2-oxo-4-(4-(6-oxo-3-phenylpyridazin-1(6H)-yl)phenyl)-2H-[1,2′-bipyridine]-3,5-dicarbonitrile, 6-Amino-2-oxo-4-(4-(6-oxo-3-phenylpyridazin-1(6H)-yl)phenyl)-1-(thiazol-2-yl)-1,2-dihydro-pyridine-3,5-dicarbonitrile, 6-amino-2-oxo-4-(4-(6-oxo-3-phenylpyridazin-1(6H)-yl)phenyl)-1-(3-(trifluoromethyl)phenyl)-1,2-dihydropyridine-3,5-dicarbonitrile, 4-(6-amino-3,5-dicyano-2-oxo-4-(4-(6-oxo-3-phenylpyridazin-1(6H)-yl)phenyl)pyridin-1(2H)-yl)benzenesulfonamide, methyl 2-(6-amino-3,5-dicyano-2-oxo-4-(4-(6-oxo-3-phenylpyridazin-1(6H) -yl)phenyl)pyridin-1(2H)-yl)benzoate, 6-amino-2-oxo-4-(4-(6-oxo-3-phenylpyridazin-1(6H)-yl)phenyl)-1-phenyl-1,2-dihydropyridine-3,5-dicarbonitrile or a pharmaceutically acceptable salt thereof.

In another embodiment of the present disclosure, a compound selected from the group is consisting of 6-amino-2-oxo-4-(4-(6-oxo-3-phenylpyridazin -1(6H)-yl)phenyl)-1-(3-(trifluoromethyl)phenyl)-1,2-dihydropyridine-3,5-dicarbonitrile, 4-(6-amino-3,5-dicyano-2-oxo-4-(4-(6-oxo-3-phenylpyridazin-1(6H) -yl)phenyl)pyridin-1(2H)-yl)benzenesulfonamide or a pharmaceutically acceptable salt thereof.

In another aspect of the present disclosure, a process for the preparation of Formula I is disclosed. The process involves reacting substituted 4-oxo-4-phenylbutanoic acid with hydrazine monohydrate to obtain substituted 6-phenyl-4,5-dihydropyridazin-3(2H)-one, followed by reacting the substituted 6-phenyl-4,5-dihydropyridazin-3(2H)-one with Bromine-AcOH to obtain substituted 6-phenylpyridazin-3(2H)-one and reacting the substituted 6-phenylpyridazin-3(2H)-one with p-fluorobenzaldehyde to produce substituted 4-(6-oxo-3-phenylpyridazin-1(6H) -yl)benzaldehyde. Finally, the process involves reacting the substituted 4-(6-oxo-3-phenylpyridazin-1(6H)-yl)benzaldehyde with substituted 2-cyanoacetamide and malononitrile to produce said Formula I.

In another aspect of the present disclosure, a process for the preparation of Formula I is disclosed. The process involves reacting the substituted 4-(6-oxo-3-phenylpyridazin-1(6H)-yl)benzaldehyde with substituted 2-cyanoacetamide and malononitrile to produce said Formula I.

One should appreciate that although the present disclosure has been explained with respect to a defined set of functional modules, any other module or set of modules can be added/deleted/modified/combined, and any such changes in the architecture/construction of the proposed system are completely within the scope of the present disclosure. Each module can also be fragmented into one or more functional sub-modules, all of which are also completely within the scope of the present disclosure.

### BRIEF DESCRIPTION OF DRAWINGS

The accompanying figures (Figs.) illustrate embodiments and serve to explain the principles of the disclosed embodiments. It is to be understood, however, that these figures are presented for purposes of illustration only and not for defining the limits of relevant applications.

FIG. 1A illustrates a chemical formula of a compound of Formula I in accordance with embodiments of the present disclosure;

FIG. 1B illustrates a reaction scheme of preparing the compound of Formula I in accordance with embodiments of the present disclosure;

FIG. 2 shows a plot of the % antioxidant activity of exemplary compounds 1-12 and ascorbic acid, in accordance with embodiments of the present disclosure;

FIG. 3 shows a plot of the % COX-2 inhibitory activity of the exemplary compounds 1-12, celecoxib, and indomethacin, in accordance with embodiments of the present disclosure; and

FIG. 4 shows a plot of the % selectivity index of the exemplary compounds 1-12, celecoxib, and indomethacin in accordance with embodiments of the present disclosure.

### TABLES

Table 01 shows antioxidant and COX inhibitory data of the exemplary compounds 1-12, in accordance with embodiments of the present disclosure; and

Table 02 shows antimicrobial activity data of the exemplary compounds 1-12, in accordance with embodiments of the present disclosure.

### DETAILED DESCRIPTION

For the purposes of promoting an understanding of the principles of the invention, reference will now be made to the exemplary embodiments illustrated in the drawings, and specific language will be used to describe the same. It will nevertheless be understood that no limitation of the scope of the invention is thereby intended. Any alterations and further modifications of the inventive features illustrated herein and any additional applications of the principles of the invention as illustrated herein, which would occur to one skilled in the relevant art and having possession of this disclosure, are to be considered within the scope of the invention.

The following is a detailed description of embodiments of the disclosure depicted in the accompanying drawings. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

Various terms, as used herein, are shown below. To the extent a term used in a claim is not defined below, it should be given the broadest definition persons in the pertinent art have given that term as reflected in printed publications and issued patents at the time of filing.

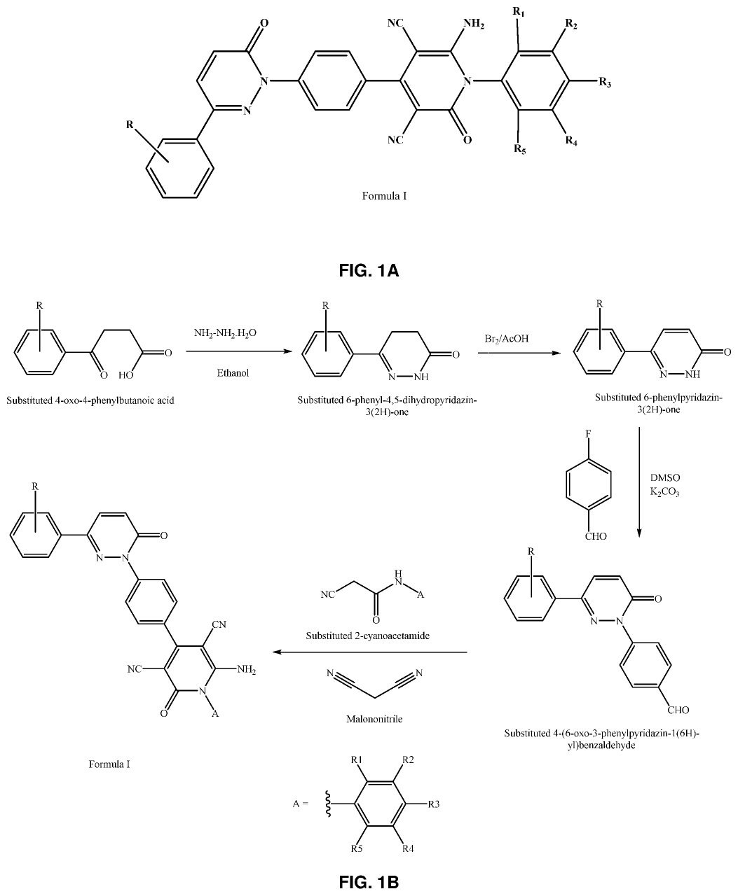

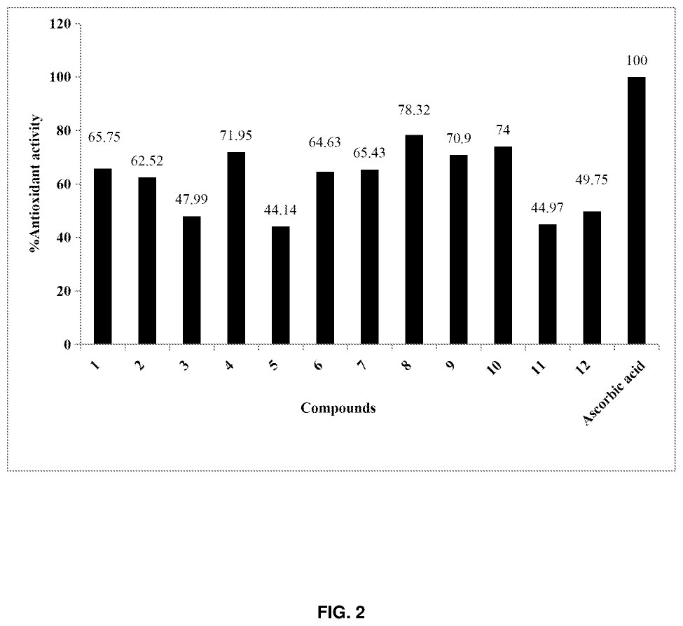

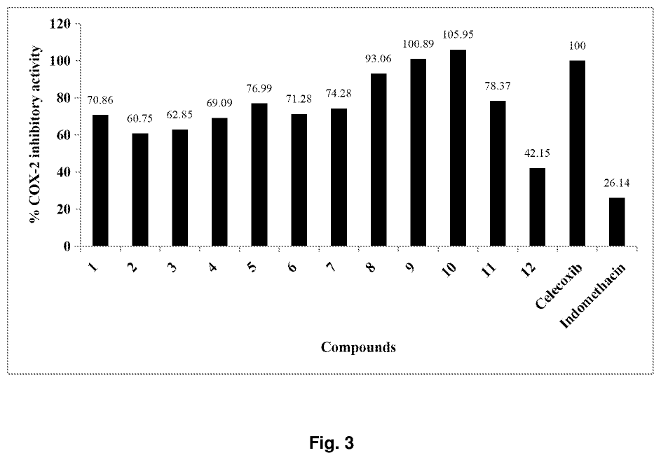

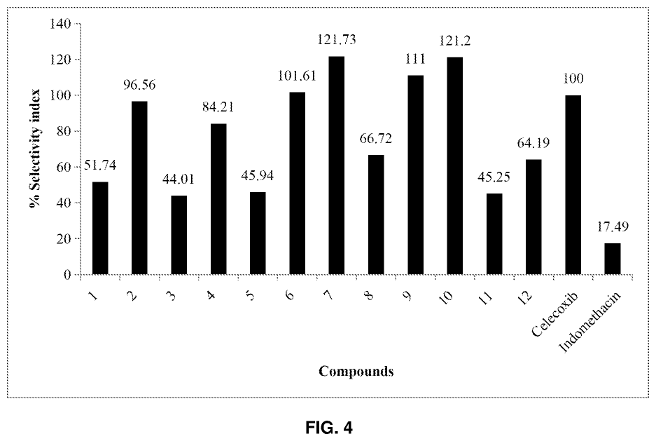

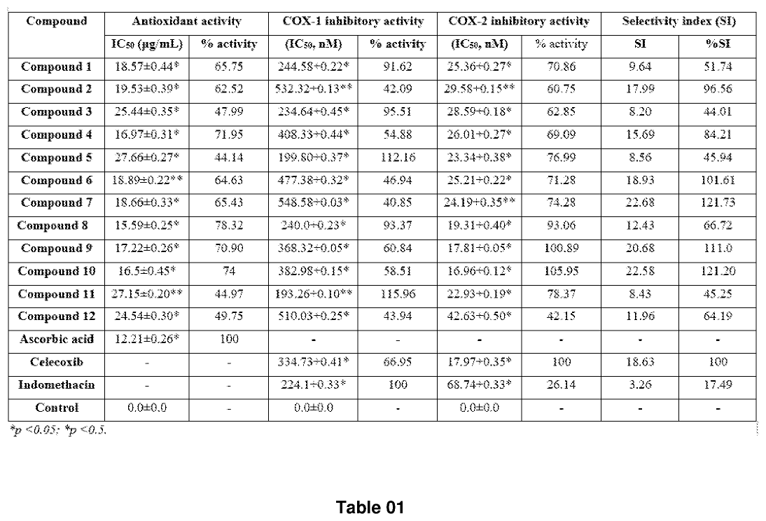

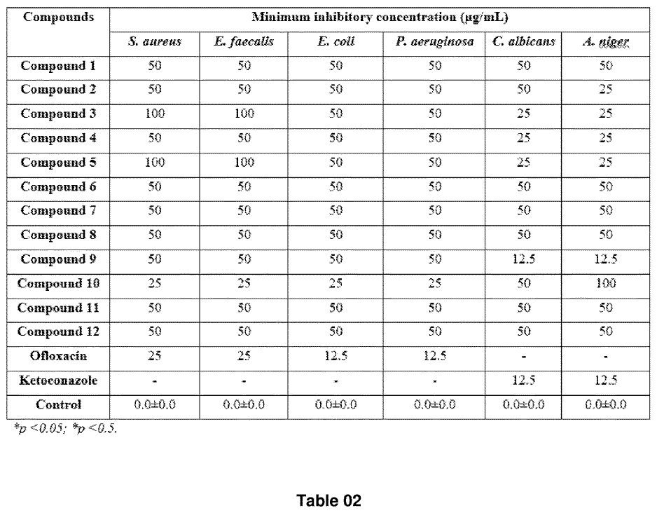

In an embodiment of the present disclosure, discloses a compound of Formula I as shown in FIG. 1A. In the Formula I, R is selected from the group consisting of —H, —F, —Cl, —Br, —I, —NO₂, —CH₃, and —C₂H₅; R₁, R₂, R₃, R₄ and R₅ are independently selected from the group consisting of —H, —COOH, -, —CONH₂, —COOCH₃, —COOCH₂CH₃, —CN, —NO₂, —OH, —CF₃, —SO₂NH₂, —F, —Cl, —Br, —I, —OCH₃, —CH₃, and —COCH₃, or a pharmaceutically acceptable salt thereof.

The compound of Formula I may be used as an active ingredient of a pharmaceutical composition for use in treating diseases associated with a higher level of ROS and COX-2 (Table 1). For example, ulcerative colitis, psoriasis, gout, osteoarthritis, ankylosing spondylitis, rheumatoid arthritis, pancreatitis, diabetes, hepatitis, irritable bowel diseases, asthma, kidney injury, ischemic stroke, atherosclerosis, neuropathic pain, epilepsy, depression, Parkinson's diseases, Crohn's disease, Alzheimer's diseases, and cancer. These compounds may also be used as an antimicrobial (Table 2).

A therapeutically effective amount of the compound of Formula I or an amount effective to treat the aforementioned diseases may be determined by in vivo assays and adjusted for the specific desired compound of Formula I using routine methods.

The compound of Formula I may be administered to a subject by any suitable route, for example, orally, nasally, rectally, parenterally, intracisternally, intravaginally, intraperitoneally, topically and transdermally. The compound may be manufactured as or incorporated into pharmaceutical compositions, including one or more of the compounds of Formula I and pharmaceutically acceptable salt thereof. The compound of Formula I may be formulated as tablets, pills, capsules, powders, granules, ointments, parenteral solutions, suspensions, aerosols, sprays, drops, ampules, auto-injector devices, suppositories, oral solutions, intranasal dosage form, sublingual dosage form or rectal dosage form or dosage form for administration by inhalation or insufflation.

In an aspect of the present disclosure, a compound of Formula II,

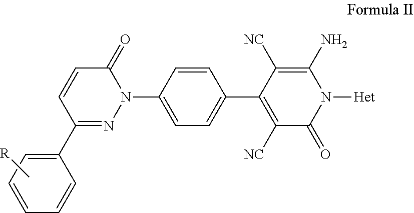

wherein R is selected from the group consisting of —H, —F, —Cl, —Br, —I, —NO₂, —CH₃, and —C₂H₅; Het is a heterocyclic ring selected from the group consisting of imidazolyl, isoxazolyl, morpholinyl, oxadiazolyl, oxazolyl, piperidinyl, pyrazinyl, pyrazolyl, pyridazinyl, pyridinyl, pyrimidinyl, quinazolinyl, quinolinyl, quinoxalinyl, quinuclidinyl, tetrazolyl, thiadiazolyl, thiazolyl, thiophenyl, triazinyl, triazolyl, indolyl, indazolyl, and pyrrolyl, wherein the heterocyclic ring may be substituted with —F, —Cl, —Br, —I, —NO₂, —CH₃, —COOH, —SO₂NH₂, —OCH₃, COOCH₃, —COOEt, —CF₃, —CHO, —COOH, —CONH₂, —OH and —CN, or a pharmaceutically acceptable salt thereof.

In an aspect of the present disclosure, a compound of Formula III,

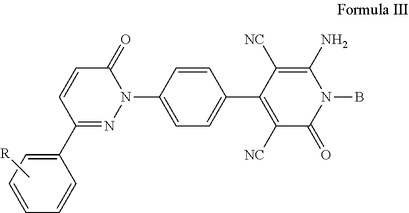

wherein R is selected from the group consisting of —H, —F, —Cl, —Br, —I, —NO₂, —CH₃, and —C₂H₅; B is a pyridinyl ring or 1,3-thiazolyl ring, wherein the pyridinyl ring and 1,3-thiazolyl ring may be substituted with —F, —Cl, —Br, —I, —NO₂, —CH₃, —COOH, —SO₂NH₂, —OCH₃, —COOCH₃, —COOEt, —CF₃, —CHO, —COOH, —CONH₂, —OH and —CN, or a pharmaceutically acceptable salt thereof.

In an aspect of the present disclosure, a compound of Formula IV, wherein R is selected from the group consisting of —H, —F, —Cl, —Br, —I, —NO₂, —CH₃, and —C₂H₅, or a pharmaceutically acceptable salt thereof.

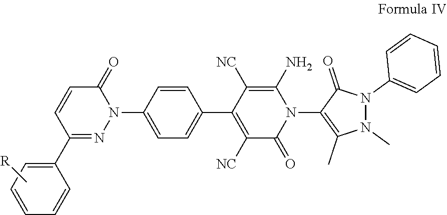

In an aspect of the present disclosure, 6-Amino-2-oxo-4-(4-(6-oxo-3-phenylpyridazin-1(6H)-yl)phenyl)-2H-[1,2′-bipyridine]-3,5-dicarbonitrile, 6-Amino-2-oxo-4-(4-(6-oxo-3-phenylpyridazin-1(6H)-yl)phenyl)-1-(thiazol-2-yl)-1,2-dihydro -pyridine-3,5-dicarbonitrile, and 6-Amino-1-(1,5-dimethyl-3-oxo-2-phenyl-2,3-dihydro-1H-pyrazol-4-yl)-2-oxo-4-(4-(6-oxo-3-phenylpyridazin-1(6H)-yl)phenyl)-1,2-dihydropyridine-3,5-dicarbonitrile.

While the present invention has been described in terms of its specific aspects and embodiments, certain modifications and equivalents will be apparent to those skilled in the art and are intended to be included within the scope of the present invention. The following examples further illustrate the present teachings.

### EXAMPLES

### Example 1: Preparation of 1-(4-Acetylphenyl)-6-amino-2-oxo-4-(4-(6-oxo-3-phenylpyridazin-1(6H)-yl)phenyl)-1,2-dihydropyridine-3,5-dicarbonitrile (Compound 1)

A mixture of 4-(6-oxo-3-phenylpyridazin-1(6H)-yl)benzaldehyde (0.01 mole), malononitrile (0.01 mole) and N-(4-acetylphenyl)-2-cyanoacetamide (0.01 mole) in ethanol (30 mL) was prepared. A few drops of piperidine were added to the mixture and refluxed for about 5 hours. The 1-(4-Acetylphenyl)-6-amino-2-oxo-4-(4-(6-oxo-3-phenylpyridazin-1(6H)-yl)phenyl)-1,2-dihydropyridine-3,5-dicarbonitrile was precipitated. The product was filtered, washed with ethanol and recrystallized from a mixture of dioxane and ethanol (1:1). Yield (78%); faint yellow solid: m.p: 218-220° C.; IR (potassium bromide, cm⁻¹): 3374 & 3216 (NH₂), 2223 & 2210 (C≡N), 1678 & 1667 (C═O); ¹H-NMR (400 MHz, DMSO-d₆; δ in ppm): 2.66 (s, 3H, CH₃), 7.25-7.27 (dd, 2H, Ar—H), 7.49-7.54 (t, 3H, Ar—H), 7.58-7.60 (dd, 2H, Ar—H), 7.71-7.73 (dd, 2H, Ar—H), 7.93-7.99 (m, 5H, 3Ar—H+NH₂), 8.16-8.19 (t, 3H, Ar—H); ¹³C-NMR (150 MHz, DMSO-d₆; δ in ppm): 198.11 (COCH₃), 160.84, 160.01, 159.14 (2C═O), 157.74, 145.11, 143.46, 135.20, 134.61, 134.50, 133.40 (2C), 131.82 (2C), 131.15 (2C), 130.99 (2C), 130.20 (3C), 129.48 (3C), 128.94, 126.66, 126.18, 116.91 (C≡N), 116.83 (C≡N), 27.42 (COCH₃); Mass (m/z): 524 (M⁺); Anal. Calcd. for C₃₁H₂₀N₆O₃: C, 70.98; H, 3.84; N, 16.02. Found: C, 70.76; H, 3.72; N, 15.87.

### Example 2: Preparation of 6-Amino-2-oxo-4-(4-(6-oxo-3-phenylpyridazin-1(6H) -yl)phenyl)-1-(p-tolyl)-1,2-dihydro-pyridine-3,5-dicarbonitrile (Compound 2)

It was prepared by following the method of example 1, wherein N-(4-acetylphenyl)-2-cyanoacetamide was replaced by 2-cyano-N-(p-tolyl) acetamide. Yield (70%); m.p: 238-240° C.; IR (potassium bromide, cm⁻¹): 3357 & 3208 (NH₂), 2218 & 2225 (C≡N), 1673 & 1664 (C═O); ¹H-NMR (400 MHz, DMSO-d₆; δ in ppm): 2.29 (s, 3H, CH₃), 7.25-7.28 (dd, 3H, Ar—H), 7.40-7.41 (dd, 2H, Ar—H), 7.48-7.55 (t, 3H, Ar—H), 7.71-7.73 (d, 3H, Ar—H), 7.92-7.99 (m, 5H, 3Ar—H+NH₂), 8.17-8.19 (d, 1H, Ar—H); ¹³C-NMR (150 MHz, DMSO-d₆; δ in ppm): 161.18, 160.08, 159.13 (2C═O), 157.76, 144.72, 144.47, 134.62, 131.47, 131.33, 129.97, 129.43 (3C), 128.62 (3C), 127.31, 126.49 (2C), 126.41 (2C), 125.83, 125.21, 116.52, 116.41 (2C≡N), 88.48, 75.71, 21.38 (—CH₃); Mass (m/z): 496 (M⁺); Anal. Calcd. for C₃₀H₂₀N₆O₂: C, 72.57; H, 4.06; N, 16.93. Found: C, 72.38; H, 3.85; N, 16.77.

### Example 3: Preparation of 6-Amino-1-(4-chlorophenyl)-2-oxo-4-(4-(6-oxo-3-phenylpyridazin-1(6H)-yl)phenyl)-1,2-dihydropyridine-3,5-dicarbonitrile (Compound 3)

It was prepared by following the method of example 1, wherein N-(4-acetylphenyl)-2-cyanoacetamide was replaced by N-(4-chlorophenyl)-2-cyanoacetamide. Yield (65%); m.p.: 241-243° C.; IR (potassium bromide, cm⁻¹): 3362 & 3225 (NH₂), 2221 & 2223 (C≡N), 1679 & 1665 (C═O); ¹H-NMR (400 MHz, DMSO-d₆; δ in ppm): 7.25-7.27 (d, 1H, Ar—H), 7.46-7.47 (d, 2H, Ar—H), 7.49-7.55 (t, 3H, Ar—H), 7.66-7.67 (dd, 2H, Ar—H), 7.70-7.73 (d, 2H, Ar—H), 7.93-7.99 (m, 5H, 3Ar—H+NH₂), 8.18-8.20 (d, 2H, Ar—H); ¹³C-NMR (150 MHz, DMSO-d₆; δ in ppm): 160.95, 160.02, 159.13 (2C═O), 157.72, 145.10, 143.47, 135.19, 134.58, 134.45, 133.39, 131.78, 131.14, 130.98 (3C), 130.19 (2C), 129.47 (2C), 128.93 (2C), 126.65 (2C), 126.19, 116.91 (2C≡N), 88.27, 75.89; Mass (m/z): 516 (M⁺); Anal. Calcd. for C₂₉H₁₇ClN₆O₂: C, 67.38; H, 3.31; N, 16.26. Found: C, 67.19; H, 3.16; N, 16.10.

### Example 4: Preparation of 6-Amino-1-(2-methoxyphenyl)-2-oxo-4-(4-(6-oxo-3-phenylpyridazin-1(6H)-yl)phenyl)-1,2-dihydropyridine-3,5-dicarbonitrile (Compound 4)

It was prepared by following the method of example 1, wherein N-(4-acetylphenyl)-2-cyanoacetamide was replaced by 2-cyano-N-(2-methoxyphenyl) acetamide. Yield (75%); m.p.: 254-256° C.; IR (potassium bromide, cm⁻¹): 3345 & 3215 (NH₂), 2222 & 2224 (C≡N), 1677 & 1662 (C═O); ¹H-NMR (400 MHz, DMSO-d₆; δ in ppm): 3.82 (s, 3H, OCH₃), 7.13-7.16 (t, 1H, Ar—H), 7.25-7.29 (q, 3H, Ar—H), 7.34-7.36 (d, 1H, Ar—H), 7.48-7.56 (t, 4H, Ar—H), 7.71-7.76 (d, 2H, Ar—H), 7.93-7.99 (m, 5H, 3Ar—H+NH₂), 8.17-8.19 (d, 1H, Ar—H); ¹³C-NMR (150MHz, DMSO-d₆; δ in ppm): 161.05, 159.43, 159.13 (2C═O), 157.51, 155.24, 145.11, 143.50, 134.58, 134.32, 132.42, 132.30, 131.78, 130.19, 129.48, 129.04 (2C), 128.71 (2C), 126.66 (2C), 126.12 (2C), 122.12, 121.93, 116.82 (C≡N), 116.14 (C≡N), 88.22, 76.39, 56.52 (—OCH₃); Mass (m/z): 512 (M⁺); Anal. Calcd. for C₃₀H₂₀N₆O₃: C, 70.30; H, 3.93; N, 16.40. Found: C, 70.15; H, 3.73; N, 16.27.

### Example 5: Preparation of Ethyl 4-(6-amino-3,5-dicyano-2-oxo-4-(4-(6-oxo-3-phenylpyridazin-1(6H)-yl)phenyl)pyridin-1(2H)-yl)benzoate (Compound 5)

It was prepared by following the method of example 1, wherein N-(4-acetylphenyl)-2-cyanoacetamide was replaced by ethyl 4-(2-cyanoacetamido)benzoate. Yield (72%); m.p.: 262-264° C.; IR (potassium bromide, cm⁻¹): 3369 & 3215 (NH₂), 2223 & 2221 (C≡N), 1719, 1674 & 1661 (C═O); ¹H-NMR (400 MHz, DMSO-d₆; δ in ppm): 1.32-1.37 (t, 3H, CH₃), 4.36-4.40 (q, 2H, CH₂), 7.25-7.27 (d, 1H, Ar—H), 7.48-7.55 (q, 3H, Ar—H), 7.58-7.60 (d, 2H, Ar—H), 7.71-7.72 (d, 2H, Ar—H), 7.93-7.99 (m, 5H, 3Ar—H+NH₂), 8.15-8.20 (dd, 4H, Ar—H); ¹³C-NMR (150 MHz, DMSO-d₆; δ in ppm): 165.71 (C═O), 160.97, 159.37, 159.03(2C═O), 157.59, 145.22, 135.56, 134.41, 132.37, 131.78, 130.35, 130.26 (4C), 129.45 (4C), 128.61, 128.13 (3C), 126.59, 126.50, 117.28 (C≡N), 116.87 (C≡N), 87.72, 75.23, 61.56 (—CH₂—), 14.64 (—CH₃); Mass (m/z): 554 (M⁺); Anal. Calcd. for C₃₂H₂₂N₆O₄: C, 69.31; H, 4.00; N, 15.15. Found: C, 69.17; H, 3.74; N, 14.98.

### Example 6: Preparation of 6-Amino-2-oxo-4-(4-(6-oxo-3-phenylpyridazin-1(6H) -yl)phenyl)-2H-[1,2′-bipyridine]-3,5-dicarbonitrile (Compound 6)

It was prepared by following the method of example 1, wherein N-(4-acetylphenyl)-2-cyanoacetamide was replaced by 2-cyano-N-(pyridin-2-yl) acetamide. Yield (68%); m.p.: 227-229° C.; IR (potassium bromide, cm⁻¹): 3362 & 3219 (NH₂), 2220 & 2218 (C≡N), 1673 & 1664 (C═O); ¹H-NMR (400 MHz, DMSO-d₆; δ in ppm): 7.25-7.27 (d, 1H, Ar—H), 7.48-7.55 (q, 3H, Ar—H), 7.60-7.65 (d, 2H, Ar—H), 7.74-7.75 (d, 2H, Ar—H), 7.94-7.99 (m, 5H, 3Ar—H+NH₂), 8.09-8.12 (t, 1H, Ar—H), 8.19-8.21 (d, 2H, Ar—H), 8.70 (d, 1H, Ar—H); ¹³C-NMR (150 MHz, DMSO-d₆; δ in ppm): 160.76, 160.33, 159.11 (2C═O), 157.01, 153.84, 147.79 (2C), 140.53, 134.59, 134.38, 131.78, 130.18, 129.47 (3C), 128.96 (2C), 126.66 (3C), 126.16 (2C), 125.03, 116.73 (C≡N), 116.45 (C≡N), 87.86, 75.80; Mass (m/z): 483 (M⁺); Anal. Calcd. for C₂₈H₁₇N₇O₂: C, 69.56; H, 3.54; N, 20.28. Found: C, 69.43; H, 3.36;N, 20.14.

### Example 7: Preparation of 6-Amino-2-oxo-4-(4-(6-oxo-3-phenylpyridazin-1(6H) -yl)phenyl)-1-(thiazol-2-yl)-1,2-dihydro-pyridine-3,5-dicarbonitrile (Compound 7)

It was prepared by following the method of example 1, wherein N-(4-acetylphenyl)-2-cyanoacetamide was replaced by 2-cyano-N-(thiazol-2-yl) acetamide. Yield (73%); m.p.: 259-261° C.; IR (potassium bromide, cm⁻¹): 3372 & 3223 (NH₂), 2214 & 2220 (C≡N), 1677 & 1661 (C═O); ¹H-NMR (400 MHz, DMSO-d₆; δ in ppm): 7.25-7.27 (d, 1H, Ar—H), 7.51-7.53 (t, 3H, Ar—H), 7.71-7.73 (d, 2H, Ar—H), 7.94-7.99 (m, 5H, Ar—H), 8.04-8.05 (d, 1H, thiazole-H5), 8.18-8.20 (d, 1H, thiazole-H4), 8.81 (s, 2H, NH₂); ¹³C-NMR (150 MHz, DMSO-d₆; δ in ppm): 166.22 (thiazole C-2), 161.15, 159.44, 159.12 (2C═O), 157.51, 155.52, 145.19 (2C), 143.57, 134.85, 134.58, 132.42, 131.79, 130.28, 129.72, 129.48, 128.97, 128.71, 126.76, 126.10, 125.34, 116.74 (C≡N), 116.56 (C≡N), 87.64, 76.61; Mass (m/z): 489 (M⁺); Anal. Calcd. for C₂₆H₁₅N₇O₂S: C, 63.80; H, 3.09; N, 20.03. Found: C, 63.72; H, 2.87; N, 19.88.

### Example 8: Preparation of 6-Amino-1-(1,5-dimethyl-3-oxo-2-phenyl-2,3-dihydro-1H-pyrazol-4-yl)-2-oxo-4-(4-(6-oxo-3-phenylpyridazin-1(6H)-yl)phenyl) -1,2-dihydropyridine-3,5-dicarbonitrile (Compound 8)

It was prepared by following the method of example 1, wherein N-(4-acetylphenyl)-2-cyanoacetamide was replaced by 2-cyano-N-(1,5-dimethyl-3-oxo -2-phenyl-2,3-dihydro-1H-pyrazol-4-yl) acetamide. Yield (70%); m.p.: 273-275° C.; IR (potassium bromide, cm⁻¹): 3362 & 3212 (NH₂), 2210 & 2215 (C≡N), 1682 & 1659 (C═O); ¹H-NMR (400 MHz, DMSO-d₆; δ in ppm): 2.18 (s, 3H, CH₃), 3.27 (s, 3H, N—CH₃), 7.24-7.26 (d, 1H, Ar—H), 7.41-7.43 (d, 3H, Ar—H), 7.51-7.56 (t, 4H, Ar—H), 7.71-7.76 (d, 3H, Ar—H), 7.93-7.98 (m, 4H, Ar—H), 8.17-8.19 (d, 1H, Ar—H), 8.60 (hump, 2H, NH₂); ¹³C-NMR (150 MHz, DMSO-d₆; δ in ppm): 161.25, 160.03, 159.24, 159.12 (3C═O), 158.35, 154.40, 145.09, 143.47, 134.85, 134.57, 132.41, 131.78, 130.18, 129.70, 129.47, 128.97 (2C), 128.71, 127.62 (2C), 126.66 (2C), 126.09, 125.33 (2C), 116.78 (C≡N), 116.25 (C≡N), 101.01 (pyrazole C-4), 87.85, 75.32, 35.41 (N—CH₃), 10.72 (—CH₃); Mass (m/z): 592 (M⁺); Anal. Calcd. for C₃₄H₂₄N₈O₃: C, 68.91; H, 4.08; N, 18.91. Found: C, 68.76; H, 3.90 N, 18.67.

### Example 9: Preparation of 6-amino-2-oxo-4-(4-(6-oxo-3-phenylpyridazin-1(6H) -yl)phenyl)-1-(3-(trifluoromethyl)phenyl)-1,2-dihydropyridine-3,5-dicarbonitrile (Compound 9)

It was prepared by following the method of example 1, wherein N-(4-acetylphenyl)-2-cyanoacetamide was replaced by 2-cyano-N-(3-(trifluoromethyl)phenyl) acetamide. 273-275° C.; IR (potassium bromide, cm⁻¹): 3364 & 3282 (NH₂), 2213 & 2217 (C≡N), 1688 & 1661 (C═O); ¹H-NMR (400 MHz, DMSO-d₆; δ in ppm): 6.78 (s, 2H, NH₂), 6.96-7.00 (d, 2H, Ar—H), 7.27-7.31 (d, 1H, Ar—H), 7.46-7.49 (t, 3H, Ar—H), 7.62-7.66 (m, 4H, Ar—H), 7.75 (s, 1H, Ar—H), 7.85-7.89 (m, 3H, Ar—H), 8.09 (d, 1H, Ar—H); ¹³C-NMR (150 MHz, DMSO-d₆; δ in ppm): 162.54 (C═O), 160.61, 159.78 (C═O), 158.74, 144.77, 134.86, 131.71, 130.96, 130.12, 129.48 (4C), 129.09 (4C), 127.31 (3C), 126.57 (3C), 126.15 (3C), 115.76 (C≡N), 115.23 (C≡N), 89.98, 77.65; Mass (m/z): 550 (M⁺); Anal. Calcd. for C₃₀H₁₇F₃N₆O₂: C, 65.45; H, 3.11; N, 15.27. Found: C, 65.28; H, 2.95; N, 15.10.

### Example 10: Preparation of 4-(6-amino-3,5-dicyano-2-oxo-4-(4-(6-oxo-3-phenylpyridazin-1(6H)-yl)phenyl)pyridin-1(2H)-yl)benzenesulfonamide (Compound 10)

It was prepared by following the method of example 1, wherein N-(4-acetylphenyl)-2-cyanoacetamide was replaced by 2-cyano-N-(4-sulfamoylphenyl) acetamide. Yield (69%); m.p.: 266-268° C.; IR (potassium bromide, cm⁻¹): 3363 & 3280 (NH₂), 2215 & 2220 (C≡N), 1684 & 1660 (C═O); ¹H-NMR (400 MHz, DMSO-d₆; δ in ppm): 7.20-7.24 (d, 2H, Ar—H), 7.46-7.49 (m, 5H, 3Ar—H+NH₂), 7.60-7.63 (d, 2H, Ar—H), 7.75-7.79 (m, 6H, Ar—H), 8.13-8.15 (d, 2H, Ar—H), 8.49-8.51 (s, 2H, NH₂); ¹³C-NMR (150 MHz, DMSO-d₆; δ in ppm): 161.17 (C═O), 160.08, 159.11 (C═O), 158.02, 145.13, 144.64, 137.73, 134.47, 131.76 (2C), 130.83 (4C), 130.23, 129.44 (3C), 126.65 (3C), 126.44 (3C), 124.96, 116.85 (C≡N), 116.25 (C≡N), 88.17, 76.84; Mass (m/z): 561 (M⁺); Anal. Calcd. for C₂₉H₁₉N₇O₄S: C, 62.03; H, 3.41; N, 17.46. Found: C, 61.84; H, 3.28; N, 17.36.

### Example 11: Preparation of methyl 2-(6-amino-3,5-dicyano-2-oxo-4-(4-(6-oxo -3-phenylpyridazin-1(6H)-yl)phenyl)pyridin-1(2H)-yl)benzoate (Compound 11)

It was prepared by following the method of example 1, wherein N-(4-acetylphenyl)-2-cyanoacetamide was replaced by methyl 2-(2-cyanoacetamido)benzoate. Yield (79%); m.p.: 260-262° C.; IR (potassium bromide, cm⁻¹): 3366 & 3284 (NH₂), 2214 & 2218 (C≡N), 1683 & 1661 (C═O); ¹H-NMR (400 MHz, DMSO-d₆; δ in ppm): 3.96 (s, 3H, OMe), 6.97 (s, 2H, NH₂), 7.26-7.30 (d, 2H, Ar—H), 7.49-7.52 (t, 3H, Ar—H), 7.55-7.58 (d, 2H, Ar—H), 7.73-8.12 (3 brs, 6H, Ar—H), 8.32-8.35 (d, 1H, Ar—H), 8.49-8.52 (s, 1H, Ar—H); ¹³C-NMR (150 MHz, DMSO-d₆; δ in ppm): 167.55 (C═O), 161.37, 160.32, 159.15 (2C═O), 158.60, 144.93, 143.08, 142.36, 139.45, 134.56, 134.05, 132.35, 131.57, 131.49, 130.22, 129.43, 128.58 (2C), 128.22 (2C), 126.49 (2C), 126.38, 126.12, 125.89, 116.93 (C≡N), 116.75 (C≡N), 87.53, 76.88, 52.88 (—OCH₃); Mass (m/z): 540 (M⁺); Anal. Calcd. for C₃₁H₂₀N₆O₄: C, 68.88; H, 3.73; N, 15.55. Found: C, 68.71; H, 3.62; N, 15.41.

### Example 12: Preparation of 6-amino-2-oxo-4-(4-(6-oxo-3-phenylpyridazin -1(6H)-yl)phenyl)-1-phenyl-1,2-dihydropyridine-3,5-dicarbonitrile (Compound 12)

It was prepared by following the method of example 1, wherein N-(4-acetylphenyl)-2-cyanoacetamide was replaced by 2-cyano-N-phenylacetamide. Yield (76%); m.p.: 280-282° C.; IR (potassium bromide, cm⁻¹): 3365 & 3280 (NH₂), 2211 & 2216 (C≡N), 1681 & 1658 (C═O); ¹H-NMR (400 MHz, DMSO-d₆; δ in ppm): 7.18-7.23 (t, 2H, Ar—H), 7.37-7.40 (t, 2H, Ar—H), 7.43-7.49 (m, 3H, 1Ar—H+NH₂), 7.59-7.61 (d, 2H, Ar—H), 7.76-7.78 (d, 2H, Ar—H), 7.86-7.93 (d, 3H, Ar—H), 8.06-8.14 (d, 3H, Ar—H), 8.24 (s, 1H, CH); ¹³C-NMR (150 MHz, DMSO-d₆; δ in ppm): 161.85(C═O), 160.32, 159.55 (C═O), 157.97, 144.59, 140.96, 137.57 (2C), 136.59, 134.65, 131.46, 131.31 (3C), 129.99 (3C), 129.38 (4C), 128.45, 126.45, 126.12, 125.53, 116.92 (C≡N), 116.34 (C≡N), 87.78, 77.64; Mass (m/z): 482 (M⁺); Anal. Calcd. for C₂₉H₁₈N₆O₂: C, 72.19; H, 3.76; N, 17.42. Found: C, 72.10; H, 3.65; N, 17.28.

### Example 13: Antioxidant Activity

The antioxidant activity was assessed by the DPPH (1,1-diphenyl-2-picryl hydrazyl) method employing ascorbic acid as a standard. A 0.1 mM solution of DPPH was freshly prepared in ethanol. Different dilutions (25, 20, 15, 10 and 5 μg/mL) of compounds 1-12 and ascorbic acid were also prepared in ethanol. The test and standard solutions were prepared by adding DPPH solution (2 mL) to the different dilutions (6 mL each) of the compounds 1-12 and ascorbic acid, respectively. The control solution was prepared by adding DPPH solution (2 mL) to ethanol (6 mL). The resultant mixtures were shaken and kept at 25° C. for half an hour. The absorbance of the test, standard, and control solutions was measured at 517 nm using a UV-Visible spectrophotometer. The experiment was performed in triplicate, and the antioxidant activity was calculated using the following equation,

$$\% Antioxidant activity = \frac{A c - A s}{A c} \times 100$$

wherein A_{c} is the absorbance of the control, and Aₛ is the absorbance of the test or standard sample. The IC₅₀ values were obtained from the regression analysis.

The antioxidant activity data of compounds 1-12 are presented in Table 01. For comparison, ascorbic acid's antioxidant activity (IC50 value) has been normalized to 100% (Table 1). The antioxidant activity data has revealed compounds 4 (71.95%), 8 (78.32%), 9 (70.90%), and 10 (74%) as moderate antioxidant compounds in comparison to ascorbic acid (100%).

### Example 14: In Vitro COX-1 and COX-2 Inhibitory Activity

The compounds 1-12 were evaluated for their ability to inhibit the COX-1 and the COX-2 enzymes (Molecules, 2020, 25 2002, https://doi.org/10.3390/molecules25092002 and Drug Development Research, 2020, 1-12, https://doi.org/10.1002/ddr.21655). The human COX-1/COX-2 test packs were obtained from Cayman Chemicals (560131, Ann Arbor, MI, USA). The supplier's instructions were followed to prepare the reagents and perform the experiment. The IC₅₀ values were determined in nmol concentration (Table 02). The IC₅₀ values of the compounds were determined from the regression analysis, wherein the selectivity index (SI) was calculated as follows.

$$Selectivity index = \frac{IC 50 for COX - 1}{IC 50 for COX - 2}$$

The COX-1 and COX-2 inhibitory data of compounds 1-12 are presented in Table 1. It is well-known that COX-2 inhibition is a therapeutic target to develop anti-inflammatory agents, whereas COX-1 inhibition causes gastric irritation or ulceration effects. Indomethacin is a non-selective COX-1 and COX-2 inhibitor and causes higher ulcerogenic effects. Celecoxib is a specific COX-2 inhibitor and is considered non-ulcerogenic. For comparison, the COX-1 inhibitory activity (IC₅₀ value) of indomethacin has been normalized to 100%; the COX-2 inhibitory activity (IC₅₀ value) of celecoxib has been normalized to 100%; and the selectivity index of celecoxib has been normalized to 100% (Table 1). A higher selectivity index value indicates more selectivity for the COX-2 enzyme. The in vitro COX inhibitory activity data has revealed compound 9 (100.89%) and compound 10 (105.95%) as the most potent COX-2 inhibitors in comparison to celecoxib (100%). The selectivity index of compound 9 (111.0%) and compound 10 (121.20%) was also better than the celecoxib (100%). It can also be observed that compound 6 (101.61%) and compound 7 (121.73%) also displayed a better selectivity index than celecoxib (100%). Interestingly and surprisingly, compounds 1-12 exhibited a better selectivity index than indomethacin (17.49%). This indicates that compounds 1-12 are more selective COX-2 inhibitors than indomethacin and may not possess the gastric ulcerating effect.

### Example 15: Antimicrobial Activity

Antimicrobial activity was performed by the serial plate dilution method (Medicinal Chemistry Research, 2017, 26, 1481-1496; Arabian Journal of Chemistry, 2016, 9, 1523-1531) against two Gram-positive bacteria, Staphylococcus aureus (S. aureus, ATCC 25923) and Enterococcus faecalis (E. faecalis, ATCC 29212); two Gram-negative bacteria, Escherichia coli (E. coli, ATCC 25922) and Pseudomonas aeruginosa (P. aeruginosa, ATCC 27853); and two fungi, Candida albicans (C. albicans, ATCC 2091) and Aspergillus niger (A. niger, MTCC 281). Antibacterial activity was performed on nutrient agar medium, whereas Sabouraud dextrose medium was used for the antifungal activity. The compounds 1-12 were tested at 100, 75, 50, 25, and 12.5 μg/mL concentrations. The standard antibiotics, ofloxacin and ketoconazole, were tested at 50, 25, and 12.5 μg/mL concentrations. Sterile dimethyl sulfoxide (DMSO) was used as a solvent for making the desired concentrations of the compounds ofloxacin and ketoconazole. Sterile DMSO also served as a control group. The minimum inhibitory concentrations (MICs) values of the synthesized compounds, ofloxacin and ketoconazole, were determined. The minimum inhibitory concentration (MIC) has been defined as the lowest concentration of a compound that inhibits the visible growth of the microorganisms in the media.

The antimicrobial activity data of compounds 1-12 are provided in Table 02. Surprisingly, it has been observed that compound 9 (MIC=12.5 μg/mL) showed equal potency with ketoconazole (MIC=12.5 μg/mL) against C. albicans and A. niger. Similarly, compound 10 (MIC=25 μg/mL) displayed equal potency with ofloxacin (MIC=25 μg/mL) against S. aureus and E. faecalis.

### Statistical Analysis

The statistical analysis was performed by the SPSS software (version 20). The experiments were performed in triplicate (N=3). The values are represented as Mean±SD (Standard Deviation). The p-value<0.05 represents the statistically significant results.

In another aspect of the present disclosure, a process for the preparation of Formula I is disclosed, as shown in FIG. 1B. The process involves reacting substituted 4-oxo-4-phenylbutanoic acid with ethanol and hydrazine monohydrate to obtain substituted 6-phenyl-4,5-dihydropyridazin-3(2H)-one, followed by reacting the substituted 6-phenyl-4,5-dihydropyridazin-3(2H)-one with Bromine-AcOH to obtain substituted 6-phenylpyridazin-3(2H)-one and reacting the substituted 6-phenylpyridazin-3(2H)-one with DMSO and Potassium carbonate to produce substituted 4-(6-oxo-3-phenylpyridazin-1(6H)-yl)benzaldehyde. Finally, the process involves reacting the substituted 4-(6-oxo-3-phenylpyridazin-1(6H)-yl)benzaldehyde with substituted 2-cyanoacetamide and malononitrile to produce said Formula I.

In another aspect of the present disclosure, a process for the preparation of Formula I is disclosed. The process involves reacting the substituted 4-(6-oxo-3-phenylpyridazin-1(6H)-yl)benzaldehyde with substituted 2-cyanoacetamide and malononitrile to produce said Formula I.

While the foregoing describes various embodiments of the invention, other and further embodiments of the invention may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

Thus, the scope of the present disclosure is defined by the appended claims and includes both combinations and sub-combinations of the various features described hereinabove as well as variations and modifications thereof, which would occur to persons skilled in the art upon reading the foregoing description.

What is claimed is:

1. A compound of Formula I 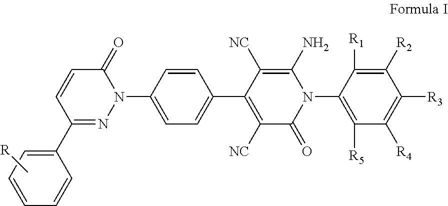
wherein R is selected from the group consisting of —H, —F, —Cl, —Br, —I, —NO₂, —CH₃, and —C₂H₅; R₁, R₂, R₃, R₄ and R₅ are independently selected from the group consisting of —H, —COOH, -, —CONH₂, —COOCH₃, —COOCH₂CH₃, —CN, —NO₂, —OH, —CF₃, —SO₂NH₂, —F, —Cl, —Br, —I, —OCH₃, —CH₃, and —COCH₃, or a pharmaceutically acceptable salt thereof.

2. The compound of Formula I of claim 1, wherein the compound is selected from selected from the group consisting of
1-(4-Acetylphenyl)-6-amino-2-oxo-4-(4-(6-oxo-3-phenylpyridazin-1(6H)-yl)phenyl) -1,2-dihydropyridine-3,5-dicarbonitrile
6-Amino-2-oxo-4-(4-(6-oxo-3-phenylpyridazin-1(6H)-yl)phenyl)-1-(p-tolyl)-1,2-dihydro-pyridine-3,5-dicarbonitrile
6-Amino-1-(4-chlorophenyl)-2-oxo-4-(4-(6-oxo-3-phenylpyridazin-1(6H)-yl)phenyl) -1,2-dihydropyridine-3,5-dicarbonitrile
6-Amino-1-(2-methoxyphenyl)-2-oxo-4-(4-(6-oxo-3-phenylpyridazin-1(6H)-yl)phenyl) -1,2-dihydropyridine-3,5-dicarbonitrile
Ethyl 4-(6-amino-3,5-dicyano-2-oxo-4-(4-(6-oxo-3-phenylpyridazin-1(6H) -yl)phenyl)pyridin-1(2H)-yl)benzoate
6-Amino-2-oxo-4-(4-(6-oxo-3-phenylpyridazin-1(6H)-yl)phenyl)-2H-[1,2′-bipyridine]-3,5-dicarbonitrile
6-Amino-2-oxo-4-(4-(6-oxo-3-phenylpyridazin-1(6H)-yl)phenyl)-1-(thiazol-2-yl)-1,2-dihydro-pyridine-3,5-dicarbonitrile
6-amino-2-oxo-4-(4-(6-oxo-3-phenylpyridazin-1(6H)-yl)phenyl)-1-(3-(trifluoromethyl)phenyl)-1,2-dihydropyridine-3,5-dicarbonitrile
4-(6-amino-3,5-dicyano-2-oxo-4-(4-(6-oxo-3-phenylpyridazin-1(6H) -yl)phenyl)pyridin-1(2H)-yl)benzenesulfonamide
methyl 2-(6-amino-3,5-dicyano-2-oxo-4-(4-(6-oxo-3-phenylpyridazin-1(6H) -yl)phenyl)pyridin-1(2H)-yl)benzoate
6-amino-2-oxo-4-(4-(6-oxo-3-phenylpyridazin-1(6H)-yl)phenyl)-1-phenyl-1,2-dihydropyridine-3,5-dicarbonitrile
or a pharmaceutically acceptable salt thereof.

3. The compound of Formula I of claim 1, wherein the compound is selected from the group consisting of
6-amino-2-oxo-4-(4-(6-oxo-3-phenylpyridazin-1(6H)-yl)phenyl)-1-(3-(trifluoromethyl) phenyl)-1,2-dihydropyridine-3,5-dicarbonitrile
4-(6-amino-3,5-dicyano-2-oxo-4-(4-(6-oxo-3-phenylpyridazin-1(6H) -yl)phenyl)pyridin-1(2H)-yl)benzenesulfonamide
or a pharmaceutically acceptable salt thereof.

4. The compound of Formula I of claim 1, wherein the compound is an active ingredient of a pharmaceutical composition for use in treating diseases associated with a higher level of ROS and COX-2.

5. The compound of Formula I of claim 1, wherein the compound is an antimicrobial agent.

6. The compound of Formula I of claim 1, wherein the compound is administered to a subject through a route selected from one or more of oral, nasal, rectal, parenteral, intracisternal, intravaginal, intraperitoneal, topical and transdermal.

7. The compound of Formula I of claim 1, wherein the compound is in a form selected from one or more of tablets, pills, capsules, powders, granules, ointments, parenteral solutions, suspensions, aerosols, sprays, drops, ampules, auto-injector devices, suppositories, oral solutions, intranasal dosage form, sublingual dosage form or rectal dosage form or dosage form for administration by inhalation or insufflation.

8. The compound of Formula I of claim 2, wherein the compound is an inhibitor of ROS and COX-2.

9. The compound of Formula I of claim 2, wherein the diseases are selected from one or more groups consisting of ulcerative colitis, psoriasis, gout, osteoarthritis, ankylosing spondylitis, rheumatoid arthritis, pancreatitis, diabetes, hepatitis, irritable bowel diseases, asthma, kidney injury, ischemic stroke, atherosclerosis, neuropathic pain, epilepsy, depression, Parkinson's diseases, Crohn's disease, Alzheimer's diseases, and cancer.

10. A process for the preparation of Formula I 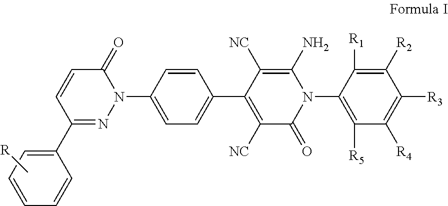
wherein R is selected from the group consisting of —H, —F, —Cl, —Br, —I, —NO₂, —CH₃, and —C₂H₅; R₁, R₂, R₃, R₄ and R₅ are independently selected from the group consisting of —H, —COOH, -, —CONH₂, —COOCH₃, —COOCH₂CH₃, —CN, —NO₂, —OH, —CF₃, —SO₂NH₂, —F, —Cl, —Br, —I, —OCH₃, —CH₃, and —COCH₃, or a pharmaceutically acceptable salt thereof,
the process comprising reacting substituted 4-(6-oxo-3-phenylpyridazin-1(6H) -yl)benzaldehyde with substituted 2-cyanoacetamide and malononitrile to produce said Formula I.

11. A process for the preparation of Formula I 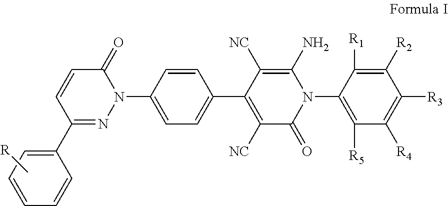
wherein R is selected from the group consisting of —H, —F, —Cl, —Br, —I, —NO₂, —CH₃, and —C₂H₅; R₁, R₂, R₃, R₄ and R₅ are independently selected from the group consisting of —H, —COOH, -, —CONH₂, —COOCH₃, —COOCH₂CH₃, —CN, —NO₂, —OH, —CF₃, —SO₂NH₂, —F, —Cl, —Br, —I, —OCH₃, —CH₃, and —COCH₃, or a pharmaceutically acceptable salt thereof,
the process comprising: 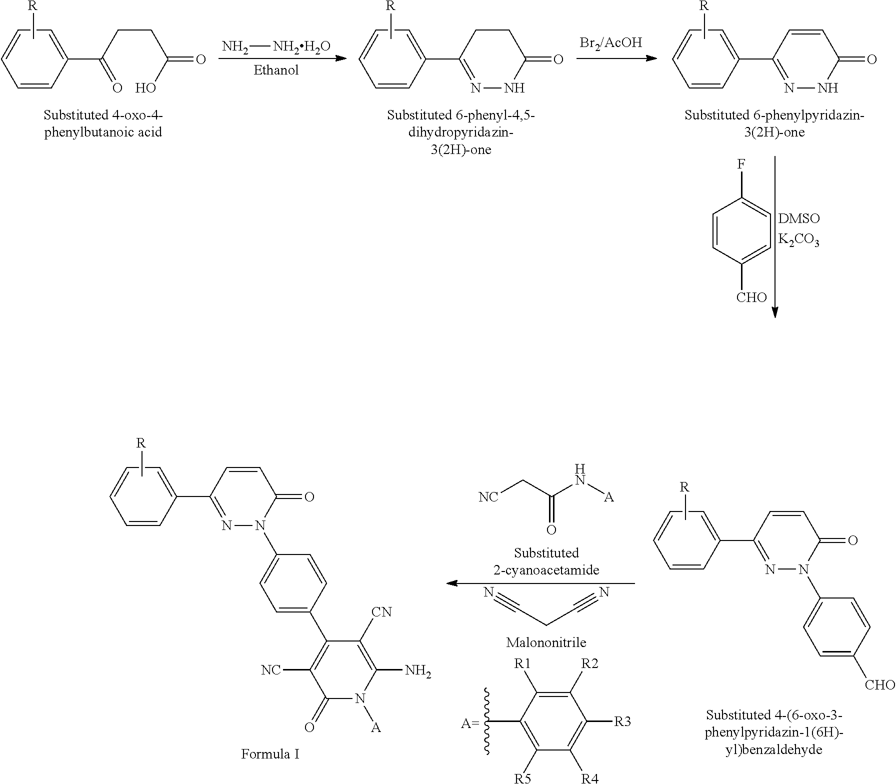
reacting substituted 4-oxo-4-phenylbutanoic acid with hydrazine monohydrate to obtain substituted 6-phenyl-4,5-dihydropyridazin-3(2H)-one;
reacting the substituted 6-phenyl-4,5-dihydropyridazin-3(2H)-one with Bromine-AcOH to obtain substituted 6-phenylpyridazin-3(2H)-one;
reacting the substituted 6-phenylpyridazin-3(2H)-one with p-fluorobenzaldehyde to produce substituted 4-(6-oxo-3-phenylpyridazin-1(6H) -yl)benzaldehyde; and
reacting the substituted 4-(6-oxo-3-phenylpyridazin-1(6H)-yl)benzaldehyde with substituted 2-cyanoacetamide and malononitrile to produce said Formula 1.
